(19) United States
(12) Patent Application Publication
(10) Pub. No.: US 2002/0096172 A1
(43) Pub. Date: Jul. 25, 2002

(54) Method and apparatus for adjusting the internal pressure of a waterproof case

(76) Inventor: Harold A. Gell II, Silver Spring, MD (US)

Correspondence Address: HAROLD GELL, P.C., 13720 LOCKDALE RD., SILVER SPRING, MD, 20906-2117, US

(21) Appl. No.: 09/681,144

(22) Filed: Jan. 25, 2001

(51) Int. Cl. B63C 11/02; A62B 7/04; A61M 16/00; F16K 31/26

(52) U.S. Cl. 128/201.27; 128/204.26

(57) ABSTRACT

An apparatus and method for controlling the pressure within a waterproof case relative to the external ambient pressure of the source of pressurizing gas. The pressurizing gas is supplied by a variable volume reservoir pneumatically connected to the case and control is maintained by positioning the case relative to the plain of the source of pressurizing gas. When the reservoir of pressurizing gas is reduced in volume by an increase in ambient water pressure to thereby increase the internal pressure of the system according to Boyle's Law, the pressure increase is coupled to the waterproof case. Thus if the waterproof case is positioned below the reservoir, the pressure within the case will be less than ambient. Conversely, if the case is positioned above the reservoir, the pressure within the case will be greater than ambient. The preferred gas reservoir is the diver's rebreather or buoyancy compensation system.

### BACKGROUND OF THE INVENTION

1. Field of the Invention

The present invention relates to a means to adjust the internal pressure within a water proof case to approximate the external ambient pressure and provide a means for minor pressure adjustment above and below the ambient pressure.

2. Discussion of the Related Art

The classical approach for solving the dilemma posed by a need to operate an apparatus underwater when the apparatus is not normally designed for immersion has been the use of a waterproof pressure hull. Such devices are bulky, costly and inordinately complex when the device has numerous controls that have to be manually activated by an operator. Some attempts have been made to solve this problem by providing a flexible, waterproof container with optical windows when necessary. Such devices may be used to encase a camera, especially the type with push-button controls where the operator may operate the camera by pressing the appropriate buttons through the fabric of the container. Unfortunately, this approach is severely limited by pressure considerations. At moderate depths, the pressure exerted on the flexible container becomes so great that push-buttons are held depressed and controls jammed. An even greater problem with this approach is that at moderate depths, the structure of the protected device becomes deformed and in many instances destroyed by the outside water pressure.

The above cited difficulties have been overcome by pressure compensation techniques such as those described in U.S. Pat. Nos. 4,771,320 and 4,771,299 for “Methodand Apparatus for Underwater Operation of Non-Waterproof Equipment”. Systems patterned in accordance with teachings of the aforementioned patents have not met with great commercial success for a variety of reasons. For instance, when a fixed volume air source such as a tether ball is used, its initial volume and the volume of the protected article limits the maximum depth to which the system may be used. Furthermore, the buoyancy of the ancillary air source for the camera case disrupts the diver's stability, i.e. the off-center buoyancy of the air source rotates the diver's body. This causes the diver to constantly compensate for the off-center buoyancy and thereby makes it extremely difficult to hold a camera steady in the weightless underwater environment, resulting in most pictures being blurred due to camera motion. The foregoing problems are minimal when very small camera cases are involved but become increasingly problematic as the size of the camera case increases or operation at greater depths is desired. Another major concern when using a fixed maximum volume air source is the absolute depth limitation imposed by the system. For instance, if the initial volume of the air source and camera case combination will permit a descent to a maximum depth of 60 feet, a desirable photograph of a creature or object located only 10 feet deeper cannot be accomplished. Another basic but significant reason is the desire of divers to minimize ancillary equipment.

### OBJECTIVES OF THE INVENTION

The underlying objective of the invention is to overcome the resistance of divers to prior pressure compensation systems by enlisting the use of a required piece of safety equipment, i.e., a buoyancy compensator or rebreather, as an air source for providing air to a waterproof camera case.

A primary objective of the invention is to provide a reservoir of air at ambient pressure for pressurizing a waterproof case.

A further objective is to provide a method for raising the internal pressure of a waterproof case relative to its ambient external pressure by adjusting the depth of the case relative to the depth of the source of pressurizing air and thereby prevent flooding of the camera case due to a defective seal or puncture.

A still further objective of the invention is to provide a reservoir of air at ambient pressure for pressurizing a waterproof case which will not affect a divers underwater center of balance.

Another objective of the invention is to provide a reservoir of air at ambient pressure for pressurizing a waterproof case wherein the reservoir may be replenished by the diver while under water to compensate for increasing depths.

Another objective of the invention is to use a diver's buoyancy compensator as a reservoir of air at ambient pressure for pressurizing a waterproof case wherein the reservoir may be replenished by the diver to compensate for increasing depths.

Another objective of the invention is to use a diver's rebreather system to provide a reservoir of air at ambient pressure for pressurizing a waterproof case.

A still further objective is to provide a method for regulating the internal pressure of a waterproof case relative to its ambient external pressure by adjusting the depth of the case relative to the depth of the source of pressurizing air.

### BRIEF SUMMARY OF THE INVENTION

According to the present invention, a waterproof case is pneumatically coupled to the normally high point of the air chamber of a diver's buoyancy compensation device or rebreather whereby the internal pressure of the case relative to its external ambient pressure may be adjusted by positioning the case above or below the high point of the air chamber.

### BRIEF DESCRIPTION OF THE SEVERAL VIEWS OF THE DRAWINGS

FIG. 1 is a perspective view of a preferred embodiment of the invention employing a buoyancy compensator as a pressurization air source.

FIG. 2 is a perspective view of an alternate embodiment of the invention wherein a buoyancy compensator inflator assembly provides a pneumatic attachment point for the pneumatic coupling to the waterproof case and the pneumatic coupling incorporates an air/water separator.

FIG. 3 is a perspective view of a preferred embodiment of the invention using a counter lung, i.e. gas bag, of a rebreather/buoyancy compensator as a pressurization air source.

FIG. 4 is a stylized schematic of a rebreather illustrating the pneumatic connection of the camera case to the rebreather ambient pressure air reservoir.

FIG. 5 illustrates the method of increasing pressure within the underwater case relative to its ambient external pressure and causing venting through a leak in the waterproof case to prevent flooding thereof .

FIG. 6 is a stylized schematic illustrating the relationship between the camera case and the air supply reservoir when the camera case is maintained at a positive relative pressure as depicted in FIG. 5.

FIG. 7 illustrates the method of increasing pressure within the underwater case relative to its ambient external pressure.

FIG. 8 is a stylized schematic illustrating the relationship between the camera case and the air supply reservoir when the camera case is maintained at a negative relative pressure as depicted in FIG. 7.

FIG. 9 is a cross-sectional view of a closed inflation needle valve of the type commonly use to inflate sports balls depicting its orientation when installed in an underwater case.

FIG. 10 is a cross-sectional view of a pneumatic connector inserted in the inflation needle valve of FIG. 9 for pneumatically connecting the underwater case to its air supply.

FIG. 11 is a cross-sectional view of a closed inflation needle valve depicting its orientation when installed in a buoyancy compensation device or rebreather.

FIG. 12 is a cross-sectional view of a pneumatic connector inserted in the inflation needle valve of FIG. 11 for pneumatically connecting a buoyancy compensation device or rebreather to the pneumatic coupling tube connected to the underwater case.

FIG. 13 is a cross-sectional view of a threaded barb for connecting the pneumatic coupling tube to a threaded port.

FIG. 14 is a cross-sectional view of a barb bonded to the buoyancy compensator, rebreater bag or waterproof case for connecting the pneumatic coupling tube.

FIG. 15 is a cross-sectional view of a threaded barb, sealing ring and nut assembly for connecting the pneumatic coupling tube to the buoyancy compensator, rebreather or waterproof case.

### DETAILED DESCRIPTION OF THE INVENTION

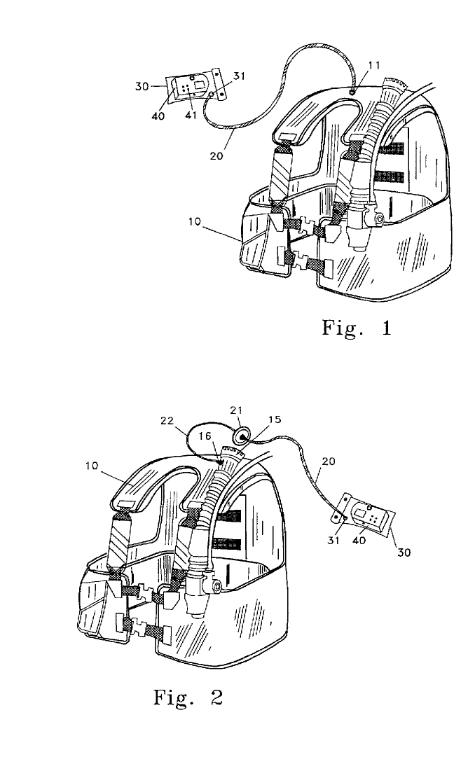

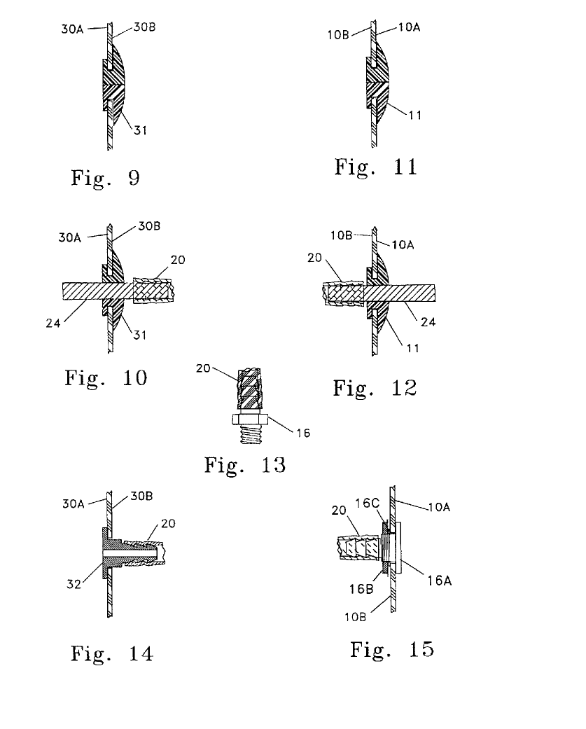

FIG. 1 illustrates a basic embodiment of the invention. A buoyancy compensator (BC) 10 includes a buoyancy chamber, air bladder, which functions as the gas reservoir for the inventive system. At least one wall of the gas reservoir is fabricated from pliant material whose response to external pressure maintains the pressure of the contents therein equal to the external ambient pressure. The air bladder includes a pneumatic port 11 which preferably is a needle inflation valve. The valve is arranged as illustrated in FIGS. 11 and 12 and positioned at what is normally the upper most section of the buoyancy chamber of the BC when a diver wearing the device is engaged in normal underwater activity. In using the invention, a diver is weighted negative so that a bubble of air must be maintained in the upper most section around the pneumatic port 11 if the diver is to achieve the neutral buoyancy required during normal diving activities. This bubble of air or gas in the reservoir is pneumatically coupled from the BC pneumatic port 11 to the underwater case 30 by a length of flexible, hollow tubing 20 which is pneumatically and mechanically secured to the underwater case by pneumatic port 31. This port may be a needle inflation valve as illustrated in FIGS. 9 and 10 but preferably is a barb with a flange bonded to the underwater case as illustrated in FIG. 14.

The invention may be used to control the pressure within any waterproof housing but in its preferred use, the waterproof housing 30 is a camera case fabricated from a pliant material dimension to fit closely about a camera 40 and thin enough to allow operation of the camera controls 41 through the walls of the case.

Preferably, it is a threaded barb such as illustrated in FIG. 13. FIG. 2 also illustrates an adaptation which may be used in any of the possible applications of the invention. This universal adaptation is the inclusion of an air/water separator 21 in the pneumatic line, hollow tub FIG. 2 illustrates an alternate embodiment wherein pneumatic port 11 is replaced by a pneumatic port 16 located in the upper section of the BC inflator assembly 15. ing 20, in FIG. 2. The air/water separator is coupled from the hollow tubing 20 to the BC by an additional section of hollow tubing 22. In alternate embodiments, the inlet end of the air/water separator is connected directly to either the air reservoir or waterproof case to eliminate the need for the extra length of tubing.

The air/water separator is a microfilter with a porosity that inhibits the flow water but allows the passage of air. One such filter that has proved successful is the Whatman L#1362. This filter uses a PTFE disk 1-¾ inches in diameter with a porosity of 0.1 Âμ. Similar filters with a smaller diameter may be used. However, the smaller the diameter of the filter disk, the smaller the amount of water required to cover its input face. When the input face of the filter disk is covered with a film water, it acts as a shut-off and air will not flow into the camera case, thus limiting the possibility of diving deeper. Rising to a shallower depth reverses the flow through filter and in some instances the reverse air flow will blow off the water film covering the input face of the filter disk, allowing a further descent. Filters using pleated filter media instead of a disk shaped filter provide significantly greater surface area and therefore can accommodate a larger volume of water before shutting off. However, such filters are more costly.

In the embodiment of the invention illustrated in FIG. 3, the recirculation system of a rebreather 12 illustrated in FIG. 3 serves as the gas reservoir. The type of rebreather illustrated in FIG. 3 uses a counter lung which is an air reservoir that provides buoyancy, thereby eliminating the need for a BC. The underwater case, camera case 30, is coupled via the hollow tubing 20 to the counter lung, air reservoir 13, of the rebreather 12.

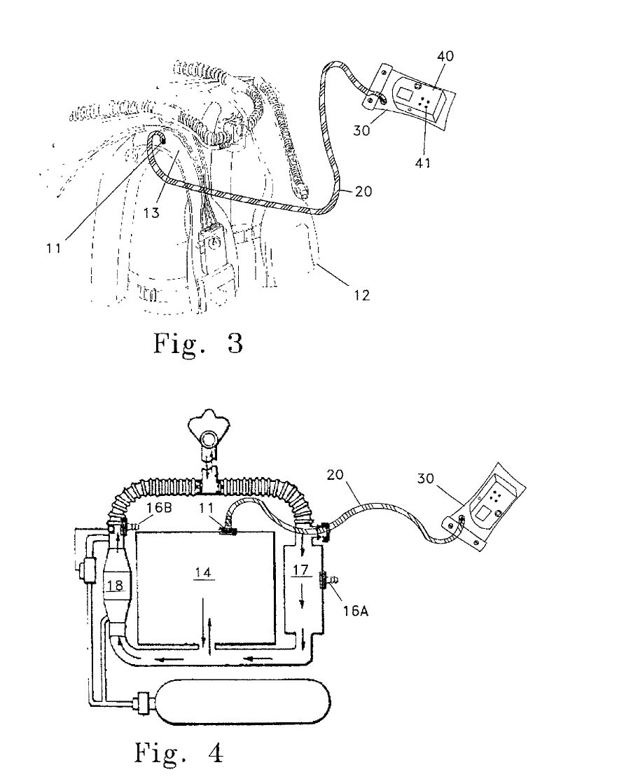

FIG. 4 is a stylized schematic view of a typical rebreather embodiment. The pneumatic line 20 connects the camera case 30 to the air reservoir 14 of the rebreather by a pneumatic port 11. The pneumatic port 11 is connected to the high point of the rebreather bag or air reservoir as in a conventional BC. However, depending on the configuration and style of the rebreather, the pneumatic port may be positioned at any convenient point along the exhalation circuit 17 as illustrated by alternate pneumatic port 16A or the inhalation circuit 18 as illustrated by the alternate pneumatic port 16B. Pneumatic ports 16A and B are threaded barbs is illustrated in FIG. 13 when the point of connection is a rigid wall section of the air circuit. Alternately, the barb may be molded as part of the rebreather hardware or bonded or bolted thereto by barbs such as illustrated in FIGS. 14 and 15. The primary consideration in positioning the pneumatic port is that a pneumatic link must be established to the ambient pressure air reservoir 14 of the rebreather.

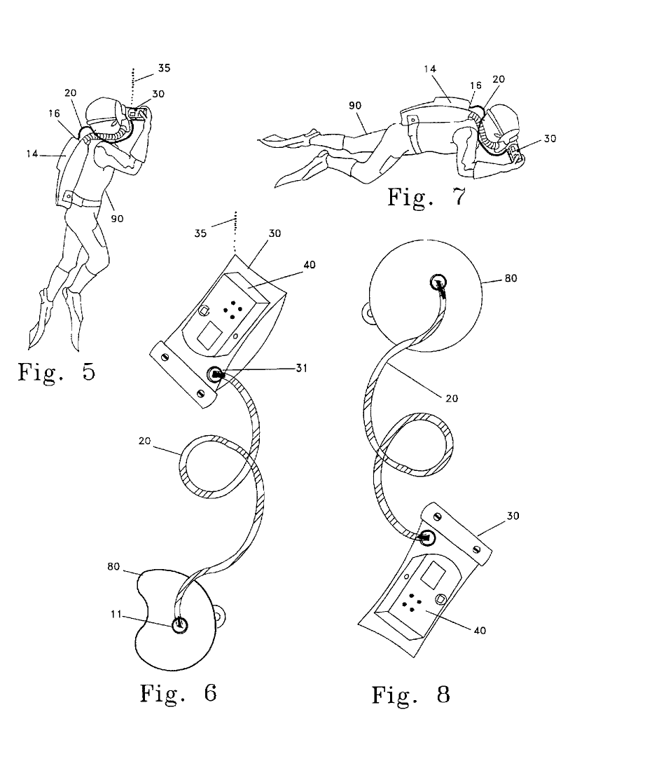

FIGS. 5 through 8 illustrates the method of the invention by which the pressure within the waterproof housing 30 is adjusted relative to the ambient water pressure. For instance, in FIG. 5, the diver 90 has assumed a vertical or near vertical position. The camera case 30 is above the high point of the air reservoir 14 of the rebreather. This results in the dynamics illustrated in FIG. 6 wherein the partially collapsed ball 80 represents the air reservoir 14. In this situation, the air that has been forced out of the ball 80 by its ambient pressure pressurizes the waterproof housing, camera case 30. With the camera case the thus pressurized by an amount equal to the pressure exerted by a column of water the height of the vertical distance between the camera case 30 and pneumatic port 16, the case balloons out from the camera. Thus the camera may be manipulated within the camera case to align the lens of the camera with the optical window of case or for any other reason. This mode of operation also tests the integrity of camera case 30.

If the camera case has a leak, the pressure within the camera case relative to its external ambient pressure results in air escaping 35 through the leak. This safety feature signals the diver that the camera case is unfit. The fact that air is flowing out of the leak prevents water from entering through the fault, thereby preventing the waterproof case from being flooded. This safety feature is available anytime during a dive. For instance, if a leak develops during the course of a dive due to a fitting loosening or the fabric of the camera case being punctured, the diver has only to hold the case above the air reservoir (i.e., ball, BC or rebreather) and flooding of the case will be prevented. This can be accomplished by simply allowing the camera case 30 to float above the air reservoir in a fashion illustrated by FIG. 6 wherein the leaking camera case is slightly inflated and released by the diver but tethered to the ball 80 by tubing 20. If the leak is large, the diver may opt to surface. If the leak is small, the diver may continue to dive with complete safety of the camera within the case so long as case is maintained above the air reservoir.

When the diver assumes a horizontal position as illustrated in FIG. 7, the air reservoir 14 and pneumatic port 16 are above the camera case 30 and the case is deflated as illustrated in FIG. 8. The ball 80 receives the air forced out of the waterproof camera case 30 by the difference in ambient external pressure associated with the ball and camera case. The camera case collapses softly about the camera, locking it in position and holding the camera lens against the optical window so that internal reflections such as might be created by a camera mounted flash are prevented. Thus an optically slaved strobe may be triggered by an integral camera flash to provide auxiliary illumination without the need for hard wiring between camera and external flash.

The preferred method of using the invention is for a diver to enter the water and descend as illustrated in FIG. 5 while holding camera case above the depth plane of the air reservoir. As the diver descends, the integrity of the waterproof case is determined by observing for the presence of escaping air in the form of bubbles. If case integrity is acceptable, the diver proceeds and adjusts the camera within the case so that the camera lens assembly is oriented properly with respect to the optical window in the case. When the camera is properly positioned, the case is lowered below the plane of the air reservoir as illustrated in FIGS. 7 and 8 to hold the optical window against the lens assembly of the camera. With the camera and case thus relatively positioned, the diver begins photographing and may trigger a flash located within the waterproof case to trigger an optically slaved strobe located outside of waterproof case without fear of internal reflections entering the lens and ruining pictures being taken. With camera and case thus relatively positioned, the case may be raised to the depth plane of the air reservoir to permit easier manipulation of the camera controls without destroying the relative positioning of the lens assembly and optical window.

Various types of pneumatic ports may be used to pneumatically couple the air reservoir and waterproof case together via the tubing 20. A preferred waterproof case pneumatic port 31 is illustrated in FIGS. 9 and 10. This device is a needle inflation valve of the type found in inflatable balls such as soccer balls, basketballs and similar sports balls. In FIG. 9 the needle is removed and the valve is closed. The domed side of the valve 31 is on the exterior 30B of the waterproof case so that external water pressure forces of valve closed as illustrated FIG. 9. FIG. 10 illustrates an inflation needle 24, which in a preferred embodiment is a stainless steel tube with a roughened exterior to assure a firm mechanical grip between the valve 31 and needle 24 and tubing 20. With the needle inserted as illustrated in FIG. 12, a pneumatic link is established between the interior of a waterproof case and the tubing 20, thereby connecting the camera case to the air reservoir via the tubing. If the needle 24 is inadvertently pulled out of the valve 30 while the system is at depth, the internal pressure generated within the camera case as the diver surfaces will cause the valve to vent excessive over pressurization. This occurs because the flat side of the valve is positioned on the inner wall 30A of the case.

In FIGS. 11 and 12, a needle inflation valve 11 is used for the air reservoir, it is oriented so that the domed surface of the valve 11 is on the inside 10A so that air will not escape from the reservoir (ball, BC or rebreather) when the inflation needle 24 is removed as illustrated in FIG. 11. When the needle 24 is inserted as illustrated in FIG. 12, a pneumatic link between the reservoir and the waterproof case is established via the tubing 20.

FIG. 13 illustrates and alternate means to connect the tubing 20 to waterproof case or air reservoir. This embodiment uses a threaded barb 16 which is screwed into a tapped hole in a rigid wall. For instance, in FIG. 2, barb 16 is threaded into a tapped hole in the rigid upper sub assembly of the inflator 15. If the invention is being used with a rebreather, a barb may be threaded into a rigid wall in the air recirculation path as illustrated by 16 A or B of FIG. 4. However, the preferred means for connecting the tubing 20 to the air reservoir is via a needle valve 11. This method of pneumatically connecting the air source is preferred because the needle 24 may be pulled free of the valve when it is not desired to use the waterproof case and the rebreather or BC will function normally without the need of ancillary hardware to close the port to the tubing 20.

Another class of embodiments uses a barb which incorporates a flange 32 that is bonded to a wall of the waterproof case or air reservoir. A still further embodiment is illustrated in FIG. 15 wherein the barb incorporates a flanged base 16A and a threaded section which receives a nut 16B whereby the barb may be secured within a hole in the waterproof case or air reservoir. An “O” ring 16C may be used to enhance sealing the barb to the wall.

The foregoing is considered as illustrative only of the principles of the invention. Further, since numerous modifications and changes will readily occur to those skilled in the art, it is not desired to limit the invention to the exact construction and applications shown and described, and accordingly, all suitable modifications and equivalents may be resorted to, falling within the scope of the invention and the appended claims and their equivalents.

What is claimed is:

1] An apparatus for adjusting the internal pressure of a waterproof case relative to the ambient external pressure, comprising: a waterproof case; a diver support system including a gas reservoir maintained at ambient pressure and selected from the class of diver support systems including buoyancy compensators and rebreathers; and means for pneumatically coupling said gas reservoir to said waterproof case.

2] An apparatus as defined by claim 1, wherein said gas reservoir includes at least one wall fabricated from pliant material which maintains the pressure of the contents therein equal to the external ambient pressure.

3] An apparatus as defined by claim 1, wherein said means for pneumatically coupling said gas reservoir to said waterproof case includes a filter with a porosity that inhibits the passage of water and allows the passage of air.

4] An apparatus as defined by claim 1, wherein said means for pneumatically coupling said gas reservoir to said waterproof case comprises: a length of flexible tubing including a waterproof case end and a gas reservoir end; a pneumatic port into said waterproof case for mechanically securing and pneumatically connecting said waterproof case end of said flexible tubing to said waterproof case; and a pneumatic port into said gas reservoir for mechanically securing and pneumatically connecting said gas reservoir end of said flexible tubing to said gas reservoir.

5] An apparatus as defined by claim 4 , wherein said pneumatic port into said gas reservoir includes a hollow barb for mechanically engaging the interior of said flexible tubing.

6] An apparatus as defined by claim 4 , wherein said pneumatic port into said waterproof case includes a hollow barb for mechanically engaging the interior of said flexible tubing.

7] An apparatus as defined by claim 4 , wherein said pneumatic port into said gas reservoir includes a sports ball needle valve and said gas reservoir end of said flexible tubing includes an inflation needle.

8] An apparatus as defined by claim 4 , wherein said pneumatic port into said waterproof case includes a sports ball needle valve and said waterproof case end of said flexible tubing includes an inflation needle.

9] An apparatus as defined by claim 4 , wherein said diver support system is a buoyancy compensator, further comprising: an inflator assembly pneumatically connected to said gas reservoir; and said pneumatic port into said gas reservoir is located in said inflator assembly.

10] An apparatus as defined by claim 4 , wherein to said diver support system is a rebreather, further comprising: an exhalation pneumatic circuit pneumatically connected to said gas reservoir; and said pneumatic port into said gas reservoir is located in said exhalation pneumatic circuit.

11] An apparatus as defined by claim 4, wherein said diver support system is a rebreather, further comprising: an inhalation pneumatic circuit pneumatically connected to said gas reservoir; and said pneumatic port into said gas reservoir is located in said inhalation pneumatic circuit.

12] An apparatus as defined by claim 4, wherein said diver support system is a buoyancy compensator containing a variable volume buoyancy compensating bladder and said gas reservoir is said variable volume buoyancy compensating bladder.

13] An apparatus as defined by claim 12, wherein said pneumatic port into said gas reservoir includes a needle valve located in said variable volume buoyancy compensating bladder and said gas reservoir end of said flexible tubing includes an inflation needle.

14] An apparatus for adjusting the internal pressure of a waterproof case relative to the ambient external pressure, comprising: a waterproof case; a diver support system including a gas reservoir maintained at ambient pressure; and means for pneumatically coupling said gas reservoir to said waterproof case.

15] A method for controlling the internal pressure of a waterproof apparatus, including the steps of: pneumatically connecting said waterproof apparatus to an inflated section of a gas reservoir which is maintained at ambient pressure and is part of a diver support system; submerging said diver support system and said waterproof apparatus; andregulating the pressure within said waterproof apparatus relative to its external ambient pressure by positioning said waterproof apparatus relative to the depth plane of said inflated section of said gas reservoir.

16] A method for controlling the internal pressure of a waterproof apparatus as defined by claim 15, including the steps of: maintaining the pressure within said waterproof apparatus equal to its external ambient pressure by maintaining the position of said waterproof apparatus at the depth of said inflated section of said gas reservoir.

17] A method for controlling the internal pressure of a waterproof apparatus as defined by claim 15, including the steps of: decreasing the pressure within said waterproof apparatus relative to the external ambient pressure of said gas reservoir by positioning said waterproof apparatus above the depth plane of said inflated section of said gas reservoir.

18] A method for controlling the internal pressure of a waterproof apparatus as defined by claim 15, including the steps of: increasing the pressure within said waterproof apparatus relative to its external ambient pressure by positioning said waterproof apparatus above the depth plane of said inflated section of said gas reservoir.

19] A method for controlling the internal pressure of a waterproof apparatus as defined by claim 18, including the steps of determining the waterproof integrity of said waterproof apparatus by maintaining said waterproof apparatus above the depth plane of said inflated section of said gas reservoir while observing for bubbles escaping from said waterproof apparatus.

20] A method for controlling the internal pressure of a waterproof apparatus as defined by claim 15, including the steps of: decreasing the pressure within said waterproof apparatus relative to its external ambient pressure by positioning said waterproof apparatus below the depth plane of said normally inflated section of said gas reservoir.

21] A method for controlling a waterproof apparatus as defined by claim 20 including the steps of: using said decreased pressure within said waterproof apparatus relative to its external ambient pressure to hold an optical window of said waterproof apparatus against the lens assembly of a camera within said waterproof apparatus, and triggering an optically slaved strobe located outside of said waterproof apparatus by firing a flash located within said waterproof apparatus.
